(19) United States
(12) Patent Application Publication
(10) Pub. No.: US 2006/0100700 A1
(43) Pub. Date: May 11, 2006

(54) Meatal occluder for closing a lachrymal meatus

(76) Inventors: Pascal Bernard, Nieul Sur Mer (FR); Philippe Tourrette, La Jarrie (FR)

Correspondence Address: YOUNG & THOMPSON, 745 SOUTH 23RD STREET, 2ND FLOOR, ARLINGTON, VA, 22202, US

(21) Appl. No.: 10/520,071

(22) PCT Filed: Jul. 1, 2003
(86) PCT No.: PCT/FR03/02036

§ 371 Date: Sep. 13, 2005

(30) Foreign Application Priority Data
Jul. 5, 2002 (FR) 02/08467

(51) Int. Cl. A61F 2/14

(52) U.S. Cl. 623/4.1

(57) ABSTRACT

A meatal occluder for closing a lachrymal meatus of a human eye includes a substantially cylindrical body (10). It further includes at least one fin (13) adapted to take up a folded position, wherein the fin (13) is substantially folded into the cylindrical body (10), and an extended position wherein the fin (13) projects from the cylindrical body (10), the fin (13) being heat-deformable from the folded position to the extended position.

The present invention relates to a meatal occluder for closing a lachrymal meatus.

This type of occluder is routinely used to occlude a lachrymal meatus of a human eye, in the pathology known as “dry eye”, which corresponds to insufficient production of lachrymal fluid by the lachrymal glands.

By inserting a meatal occluder into each lachrymal meatus, in the upper and lower part of the corner of each eye, the natural evacuation of the lachrymal fluid may be regulated to prevent dehydration of the eyes.

A device of the above kind is described in particular in the document U.S. Pat. No. 6,234,175. The small device described in the above document takes the form of a narrow cylinder having a diameter suitable for insertion of the device into the canalicule extending the lachrymal meatus.

The device is made of heat-sensitive acrylic so that it is softened and deformed isovolumetrically when heated to a temperature above its vitreous transition temperature.

When the device is placed in the lachrymal meatus, it responds to the rise in temperature and expands in the widthwise direction to adapt to the size and to the shape of the canalicule. However, it is still possible for the device to migrate either inside the canalicule, in the direction of the nasal fossa, or to the exterior, since the cylindrical shape of the device is unable to guarantee that it is retained in position.

An object of the present invention is to overcome the problems referred to above and to propose a meatal occluder that is retained in position inside the lachrymal meatus in a particularly reliable manner.

The present invention is therefore directed to a meatal occluder for closing a lachrymal meatus of a human eye that comprises a substantially cylindrical body.

According to the invention, the meatal occluder comprises at least one fin adapted to take up a folded position, wherein the fin is substantially folded into the cylindrical body, and an extended position, wherein the fin projects from the cylindrical body, said fin being heat-deformable from said folded position to said extended position.

Thus the folded position of the fins preserves the cylindrical shape of the occluder, thereby facilitating its insertion into the lachrymal meatus by the surgeon.

Inside the lachrymal meatus, the heat-deformable fin is extended because of the thermal effect generated by the human body. This projecting position of the fin holds the occluder in position inside the lachrymal meatus and thereby prevents its expulsion.

According to one preferred feature of the invention, the occluder is made of a heat-expandable material so that the deformation of the fin may be readily obtained as soon as the occluder is placed in the canalicule, because of the thermal effect of the human body.

According to one particularly practical feature of the invention, said fin pivots between said folded position and said extended position about an axis perpendicular to a longitudinal plane of said meatal occluder.

The fins may therefore be extended in the manner of an umbrella, forming projections around the cylindrical occluder adapted to come into contact with the inside wall of the canalicule in which the occluder is placed.

According to another preferred feature of the invention, said fin is situated in the vicinity of a tapered end of said cylindrical body, the opposite end of said cylindrical body comprising a flange.

Thus the meatal occluder may be held in position at both ends, to prevent both its expulsion from the lachrymal meatus and its migration deeper into the canalicule.

According to one particularly advantageous feature of the invention, reinforcing the retention of the occluder in position, the occluder comprises a plurality of fins regularly distributed on the cylindrical body of said occluder.

Other features and advantages of the invention will emerge further in the following description.

In the appended drawings, which are provided by way of nonlimiting example:

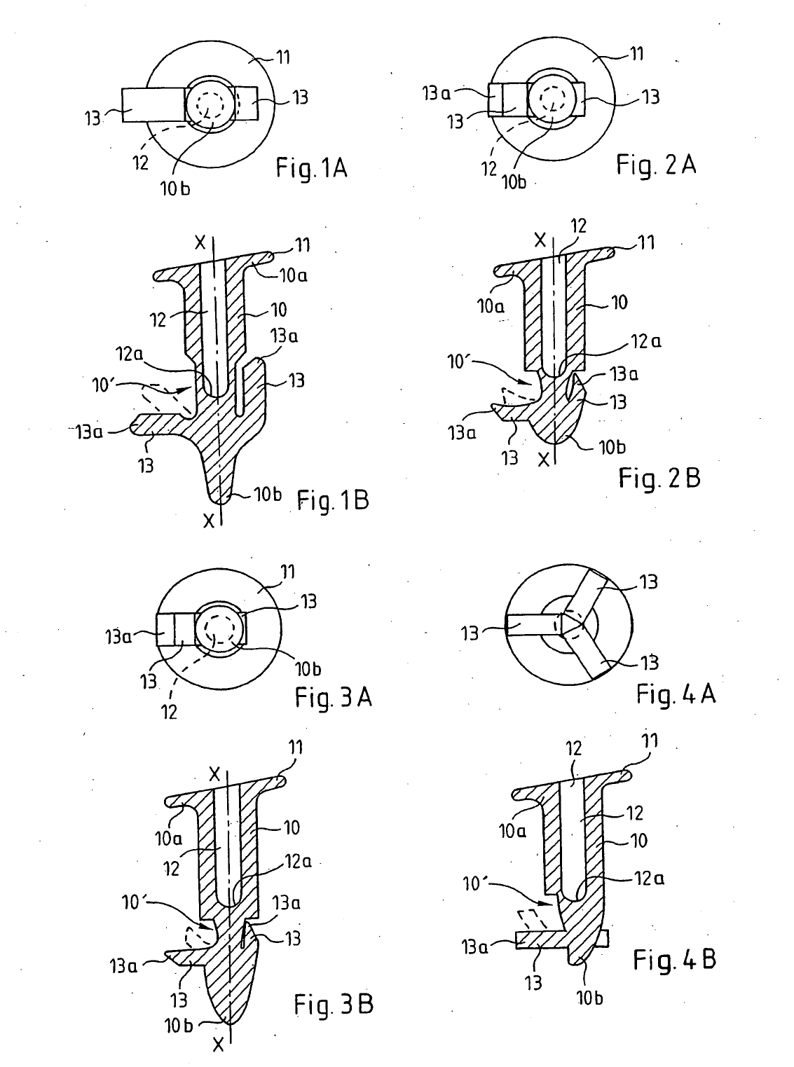

FIG. 1A is a bottom view of a meatal occluder conforming to a first embodiment of the invention;

FIG. 1B is a view in longitudinal section of the FIG. 1A meatal occluder;

FIG. 2A is a bottom view of a meatal occluder conforming to a second embodiment of the invention;

FIG. 2B is a view in longitudinal section of the FIG. 2A meatal occluder;

FIG. 3A is a bottom view of a meatal occluder conforming to a third embodiment of the invention;

FIG. 3B is a view in longitudinal section of the FIG. 3A meatal occluder;

FIG. 4A is a bottom view of a meatal occluder conforming to a fourth embodiment of the invention;

FIG. 4B is a view in longitudinal section of the FIG. 4A meatal occluder;

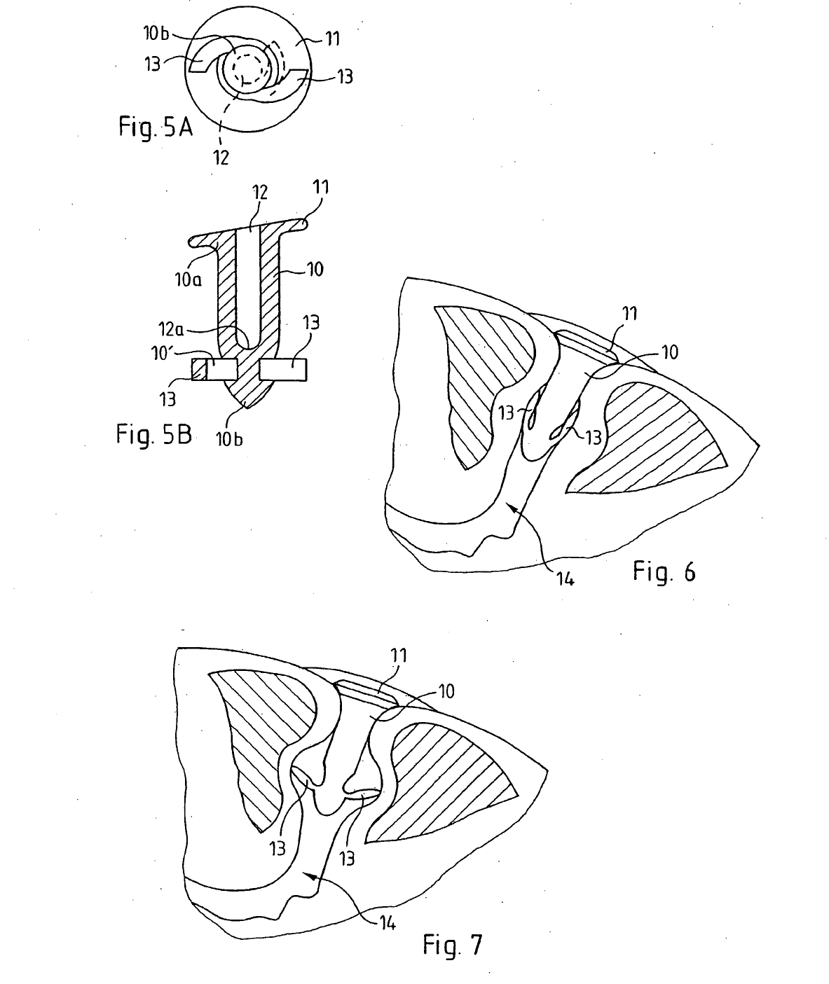

FIG. 5A is a bottom view of a meatal occluder conforming to a fifth embodiment of the invention;

FIG. 5B is a view in longitudinal section of the FIG. 5A meatal occluder; and

FIGS. 6 and 7 are diagrammatic views showing the positioning of the meatal occluder in the lachrymal meatus, respectively with the fins folded and extended.

A first embodiment of a meatal occluder conforming to the invention is described first with reference to FIGS. 1A and 1B.

In a manner that is known in the art, this meatal occluder is used to close a lachrymal meatus of a human eye in order to limit the loss of lachrymal liquid.

The meatal occluder comprises a substantially cylindrical body 10 having a flange 11 at a first end 10a.

The longitudinal direction of the cylindrical body extends along an axis X.

The cylindrical body 10 is bored along its longitudinal direction X with the result that it comprises a longitudinal orifice 12, also of substantially cylindrical shape, that opens from the body 10 at its first end 10a, inside the flange 11.

The longitudinal orifice 12 stops short of a second end 10b of the cylindrical body and therefore has a closed bottom 12a inside the cylindrical body 10.

In a manner that is known in the art, this longitudinal orifice 12 inside the meatal occluder is used to insert an occluder fitting device used to place the meatal occluder in the lachrymal meatus.

To facilitate the insertion of the meatal occluder into the lachrymal meatus, the second end 10b of the cylindrical body is tapered. This tapered end 10b of the occluder is the end that is inserted into the lachrymal meatus.

In this embodiment, the meatal occluder comprises two fins 13 regularly distributed on the cylindrical body 10 of the occluder, i.e. disposed along a diameter of the occluder.

As is clearly shown, each fin 13 is adapted to occupy a folded position, wherein the fin 13 is retracted inside the cylindrical body 10 (see the right-hand part of FIGS. 1A and 1B), and an extended position, wherein the fin 13 projects from the cylindrical body 10 (see the left-hand part of FIGS. 1A and 1B).

In order to explain the invention, one of the fins is shown in the extended position and the other in the folded position; in reality, the fins 13 are either both folded or both extended, of course.

The fins 13 pivot between their folded position and their extended position about an axis perpendicular to a longitudinal plane of the occluder, i.e. an axis perpendicular to the plane of FIG. 1B.

In their folded position, the fins 13 extend in a direction substantially parallel to the longitudinal direction X of the cylindrical body 10, so that the insertion of the tapered end 10b of the occluder is not impeded by the presence of the fins.

Here, each fin 13 is situated in the vicinity of the second end 10b of the cylindrical body 10, the free end 13a of each fin 13 when in the folded position extending in the direction of the first end 10a of the cylindrical body.

In their extended position, the fins 13 extend substantially in a transverse plane of the cylindrical body 10 of the occluder.

A second embodiment of a meatal occluder conforming to the invention is described next with reference to FIGS. 2A and 2B.

In these and all subsequent figures, elements common to the first embodiment carry the same reference numbers and do not need to be described in detail.

In this second embodiment, the occluder also comprises two fins 13 adapted to pivot between a folded position and an extended position around an axis perpendicular to a longitudinal plane of the occluder, i.e. an axis perpendicular to the plane of FIG. 2B.

In this embodiment, the free end 13a of each fin is tapered.

Unlike the first embodiment, in which the free end 13a of each fin 13 extends along the cylindrical body 10 beyond the bottom 12a of the longitudinal orifice 12, in this second embodiment the end 13a of each fin 13 terminates substantially at the level of the transverse plane coinciding with the bottom 12a of the longitudinal orifice 12.

Furthermore, the second end 10b of the cylindrical body 10 is not tapered, as such, but hemispherical.

A third embodiment of a meatal occluder conforming to the invention is described next with reference to FIGS. 3A and 3B.

This occluder also has two fins 13 adapted to be extended by pivoting about an axis perpendicular to the longitudinal plane of the occluder.

As in the second embodiment, the ends 13a of each fin 13 are tapered.

Unlike the first and second embodiments, the free end 13a of each fin 13 terminates short of the bottom 12a of the longitudinal orifice 12 of the occluder.

In each of the above embodiments, the cylindrical body 10 of the occluder comprises a portion 10′ of reduced diameter inside which the fins 13 lie in their folded position.

A fourth embodiment of a meatal occluder conforming to the invention is described next with reference to FIGS. 4A and 4B.

Unlike the first three embodiments, this occluder comprises three fins regularly distributed on the cylindrical body 10 of the occluder.

The fins 13 are therefore disposed at 120° to each other.

As previously, the fins are adapted to pivot about an axis perpendicular to the longitudinal plane of the occluder between a folded position, retracted inside a reduced diameter portion 10′ of the cylindrical body 10, and an extended position.

In this extended position, each fin 13 lies in a plane transverse to the longitudinal direction X of the occluder 10.

As previously, the cylindrical body 10 of the occluder has a flange at a first end 10a and a tapered second end 10b.

In this embodiment with three fins 13, the free end 13a of each fin extends substantially as far as the transverse plane of the occluder corresponding to the bottom 12a of the longitudinal orifice 12 provided inside the cylindrical body 10 for inserting an occluder fitting device.

Finally, a fifth embodiment of a meatal occluder conforming to the invention is described with reference to FIGS. 5A and 5B.

This meatal occluder again comprises two fins 13.

Unlike the preceding embodiments, these fins 13 are adapted to pivot between their folded position and their extended position about an axis parallel to the longitudinal direction X of the cylindrical body 10 of the occluder.

The fins 13 therefore move in a common transverse plane of the cylindrical body 10 situated between the bottom 12a of the longitudinal orifice of the occluder and the tapered end 10b of the occluder.

In the folded position, the fins 13 are accommodated inside a reduced diameter portion 10′ of the cylindrical body 10.

In all the foregoing embodiments, the meatal occluder may be made from a heat-expandable material so that the fins 13 are extended by the action of heat.

Thus each fin 13 is heat-deformable from its folded position to its extended position.

The materials from which the occluder may be made may be chosen from different types of polymers, homopolymers, cross-linked polymers, silicones, acrylic polymers, polyurethanes and hydrocarbonated polymers.

Of course, the occluder may equally be made from a combination of the foregoing polymers.

The above polymers are biologically inert, biocompatible, and non-immunogenic.

These polymers may be grouped into two classes, distinguished by their melting point Tf of their vitreous transition temperature Tg.

Polymers having a vitreous transition temperature Tg from −10° C. to 30° C., and in any event less than or equal to 37° C., include in particular silicones, acrylics, polyurethanes, hydrocarbonated polymers and their copolymers.

These copolymers may be formulated with waxes such as octadecane or oligomers of polyethylene to increase their stiffness at temperatures below the vitreous transition temperature of the material.

By way of nonlimiting example, a mixture of lauryl methacrylate in a proportion of 40% by weight and methyl methacrylate in a proportion of 60% by weight yields a copolymer having a vitreous transition temperature of the order of 19° C.

Polymers having a melting point Tf less than or equal to 37° C. may be used instead. For example, acrylic polymers having a long side chain able to crystallize may be used in this application.

By way of nonlimiting example, a stearyl polymethacrylate having a melting point of the order of 34° C. may be used.

A finned occluder of the above kind may be injection molded at low pressure.

A twin-shell multi-imprint mold is assembled in a sealed manner. After mixing the various monomers and other constituents, the composition may be injected into the mold. The mold is then placed in a temperature-controlled oven for a period from 5 to 72 hours to polymerize the materials.

After polymerization, the mold is disassembled and the meatal occluder is removed from its imprint.

Molding stalks are generally cut off by hand and a visual inspection may be carried out to eliminate occluders that do not satisfy predefined quality criteria.

Depending on the type of polymers used, the occluder may be heated to a temperature above its vitreous transition temperature Tg or its melting point Tf and the fins folded along the body of the occluder, into the reduced diameter portion 10′ of the cylindrical body 10, in order to reduce the overall size.

The occluder is then cooled by a jet of cooled air and placed on an occluder fitting device by means of the longitudinal orifice 12.

A piece of non-deformable tube, for example a thick-walled silicone tube, is placed around the occluder, forming a sleeve to prevent the fins 13 extending before use, even if the recommended storage temperature is temporarily exceeded.

Otherwise, exceeding the recommended storage temperature, and in particular an increase in the temperature above 37° C., would instantaneously cause the fins 13 to extend.

The occluder may then be placed in a blister pack and sterilized.

As shown clearly in FIGS. 6 and 7, the occluder may be inserted into the lachrymal meatus at room temperature with the fins 13 in the folded position.

Thus, during insertion, the occluder of the invention has a substantially cylindrical shape, facilitating its insertion into the lachrymal meatus and then into the canalicule 14.

As soon as the meatal occluder is in position inside the canalicule 14, the heat-deformable fins 13 resume their extended position, because of the heating effect of the human body, coming into close contact with the inside wall of the canalicule.

The extended fins 13 therefore hold the occluder reliably in position inside the canalicule.

Of course, numerous modifications may be made to the embodiments described hereinabove without departing from the scope of the invention.

1. A meatal occluder for closing a lachrymal meatus of a human eye, comprising a substantially cylindrical body (10) and characterized in that it further comprises at least one fin (13) adapted to take up a folded position, wherein the fin (13) is substantially folded into the cylindrical body (10), and an extended position wherein the fin (13) projects from the cylindrical body (10), said fin (13) being heat-deformable from said folded position to said extended position.

2. Meatal occluder according to claim 1, characterized in that it is made from a heat-expandable material.

3. Meatal occluder according to claim 1, characterized in that it is made from polymers chosen from a group comprising polymers, homopolymers, cross-linked polymers, silicones, acrylic polymers, polyurethanes, hydrocarbonated polymers and a combination of the above polymers.

4. Meatal occluder according to claim 2, characterized in that the heat-expandable material has a vitreous transition temperature from −10° C. to 30° C.

5. Meatal occluder according to claim 1 characterized in that said fin (13) pivots between said folded position and said extended position about an axis perpendicular to a longitudinal plane of said meatal occluder.

6. Meatal occluder according to claim 5, characterized in that said fin (13) when in said folded position extends in a direction substantially parallel to the longitudinal direction (X) of the cylindrical body (10).

7. Meatal occluder according to claim 5, characterized in that said fin (13) is situated in the vicinity of one end (lob) of said cylindrical body (10), a free end (13a) of said fin (13), when in the folded position, extending in the direction of the opposite end (10a) of said cylindrical body (10).

8. Meatal occluder according to claim 1 characterized in that said fin (13) pivots between said folded position and said extended position about an axis parallel to the longitudinal direction (X) of the cylindrical body (10).

9. Meatal occluder according to claim 1, characterized in that said fin (13) is situated in the vicinity of a tapered end (10b) of said cylindrical body (10), the opposite end (10a) of said cylindrical body (10) comprising a flange (11).

10. Meatal occluder according to claim 1, characterized in that it comprises a plurality of fins (13) regularly distributed on the cylindrical body (10) of said occluder.
